(19) United States
(12) Patent Application Publication
(10) Pub. No.: US 2009/0130714 A1
(43) Pub. Date: May 21, 2009

(54) Process for purifying recombinanat tissue plasminogen activator (TPA)

(75) Inventors: Sudip Kumar Majumder, Maharashtra (IN); Gita Arvind, Maharashtra (IN); Bhavesh Saxena, Maharashtra (IN)

Correspondence Address: FOLEY AND LARDNER LLP;SUITE 500, 3000 K STREET NW, WASHINGTON, DC, 20007, US

(73) Assignee: Reliance Life Sciences PVT.LTD.

(21) Appl. No.: 12/232,746

(22) Filed: Sep. 23, 2008

(30) Foreign Application Priority Data
Sep. 24, 2007 (IN) 1857/MUM/2007

(51) Int. Cl. C12P 21/02

(52) U.S. Cl. 435/69.1

(57) ABSTRACT

The present invention relates to an efficient and improved process for purifying a recombinant protein. The invention relates to the purification of tissue plasminogen activator (tPA), such as truncated human tPA, recombinantly produced in bacteria, for example in E. coli. The present invention provides a process that requires less refolding volume after solubilization of inclusion bodies isolated from cells expressing the recombinant tPA, without affecting the yield and purity of the tPA protein. The invention also provides optimum arginine concentrations for use during protein refolding and during ion exchange chromatography.

### CROSS-REFERENCE TO RELATED PATENT APPLICATIONS

This application claims benefit of provisional Indian Application No. 1857/MUM/2007, filed Sep. 24, 2007, which is hereby entirely incorporated by reference.

### FIELD OF THE INVENTION

The present invention relates to a simple and efficient process for the purification of recombinant tissue plasminogen activator (tPA) protein. In one embodiment, the invention relates to the purification of recombinantly expressed tPA, such as human truncated tPA, wherein the process optimizes arginine concentration during chromatography.

### BACKGROUND OF THE INVENTION

Thrombosis is an important part of a normal hemostatic response that limits hemorrhage from microscopic or macroscopic vascular injury. Physiologic thrombosis is counterbalanced by intrinsic antithrombotic properties and physiologic fibrinolysis. Under normal conditions, the thrombus is confined to the immediate area of injury and does not obstruct flow to critical areas, unless the vessel lumen is already diminished, such as in atherosclerosis. Under pathological conditions, a thrombus can propagate into otherwise normal vessels. A thrombus that has propagated where it is not needed can obstruct flow in critical vessels and can obliterate valves and other structures that are essential to normal hemodynamic function. The principal clinical syndromes that can result are acute myocardial infarction (MI), deep vein thrombosis, pulmonary embolism, acute ischemic stroke, acute peripheral arterial occlusion, and occlusion of indwelling catheters.

Both hemostasis and thrombosis depend on the coagulation cascade, vascular wall integrity, and platelets response. Several cellular factors are responsible for thrombus formation. When a vascular insult occurs, an immediate local cellular response takes place. Platelets migrate to the area of injury where they secrete several cellular factors and mediators. These mediators promote thrombus formation.

During thrombus formation, circulating prothrombin is activated by platelets. In this process, fibrinogen also converts to fibrin, which then creates the fibrin matrix. All this takes place while platelets are being adhered and aggregated. Thrombolytic drugs convert fibrin-bound plasminogen to plasmin, the rate-limiting step in thrombolysis.

A thrombolytic drug breaks up or dissolves blood clots, which are the main cause of both heart attacks and stroke. By dissolving the clot, the blood is able to start flowing again to that area of the heart, brain, or other organ affected by thrombosis. If blood flow to the heart is started again rapidly, it may prevent long-term damage to the heart muscle and may even stop an event that could be fatal. Thrombolytic agents available today are serine proteases that work by converting plasminogen to the natural fibrinolytic agent plasmin. Plasmin lyses clots by breaking down the fibrinogen and fibrin contained in a clot. Urokinase like plasminogen activators are produced in renal cells. They circulate in blood and are excreted in the urine. Their ability to catalyze the conversion of plasminogen to plasmin is affected slightly by the presence or absence of a local fibrin clot.

No single plasminogen activator has been approved by the U.S. Food and Drug Administration (FDA) to be labeled for every indication. New agents and new dosing regimens are under constant investigation. A choice of plasminogen activator is generally based upon the results of ongoing clinical trials and upon the clinician's experience. The most appropriate agent and regimen for each clinical situation changes over time and may differ from patient to patient.

Three groups of thrombolytic agents are available, including (i) enzymes, which act directly upon the fibrin strands within the clot, (ii) plasma activator agents, which increase plasma activator activity, and (iii) plasminogen activators, such as streptokinase, urokinase, and tissue plasminogen. All of these drugs digest clots by increasing the amount of plasmin in the blood. Plasmin, a serine protease, dissolves clots. To produce plasmin, the substance plasminogen must first be activated. Plasminogen is converted into plasmin by certain enzymes known as plasminogen activators.

One plasminogen activator, streptokinase, has been used since about 1960. Researchers use streptococci bacteria to produce this drug. Although streptokinase is the least expensive activator, some negative side effects, such as immune responses, have been experienced by patients. Another plasminogen activator, urokinase, is found naturally in humans, especially in the urine. Thus, no negative immune response is associated with its use. Because it is difficult to purify, and therefore rather expensive, however, this therapy is usually administered in small doses and combined with other drugs.

Tissue plasminogen activator (tPA) is also currently used for dissolving blood clots. It is unique because it activates only fibrin-bound plasminogen and thus targets the clot site. tPA in human blood is produced in very small amounts by vascular endothelial cells.

tPA is a secreted serine protease that converts the proenzyme plasminogen to plasmin, a fibrinolytic enzyme. Plasminogen is synthesized as a single chain that is cleaved by tPA into the two chain disulfide linked plasmin. Plasmin plays a role in cell migration and tissue remodeling. Increased enzymatic activity causes hyperfibrinolysis, which manifests as excessive bleeding; decreased activity leads to hypofibrinolysis which can result in thrombosis or embolism. Thus, tPA is an enzyme that helps dissolve clots. tPA is produced by the cells lining blood vessels and has also been made in the laboratory. It is a systemic thrombolytic (clot-busting) agent and is used in the treatment of heart attack and stroke.

tPA is known to be secreted naturally from a number of tissue sources including heart, fetal kidney, lung, and colon fibroblast cells. tPA has been previously produced using recombinant means by a number of groups, initiated by the successful cloning of the cDNA by Pennica et al., Nature, 301: 214-221 (1983). The tPA protein can be recombinantly produced in a variety of hosts including E. coli, mouse L cells, CHO cells and yeast. See, e.g., EP 174,835 (UpJohn), EP 161,935 (Eli Lilly), EP 143,081 (Ciba-Geigy), WO86/05514 (Chiron), EP 117,059 (Genentech) and EP 117,060 (Genentech). Native tPA has been isolated according to methods as described in Snow Brand Milk Products (EP 196,226); Kochi Medical School (EP 194,736); Kowa K K and Asahi (EP 151,996); Meiji Milk Products (GB 2,153,366); Choay, S. A. (EP 133,070); Asahi and Kowa K K (U.S. Pat. No. 4,505,893); Wakamoto Pharmaceutical (Biotechnology, November 1986).

Overexpression of recombinant proteins, such as tPA, in E. coli often leads to accumulation in the form of insoluble aggregates known as inclusion bodies. Inclusion bodies are composed of densely packed denatured protein molecules in the form of particles. To obtain active recombinant protein, the inclusion bodies must be solubilized and then soluble monomeric protein must be refolded into a bioactive form. Refolding of inclusion body derived proteins, however, is cumbersome, results in poor recovery and accounts for the major cost in production of recombinant proteins from E. coli.

Until recently, tPA has been purified using various types of affinity chromatography. For example, tPA recombinantly produced in E. coli as inclusion bodies, and then solubilized, is generally purified after refolding with the help of lysine affinity chromatography. Other affinity chromatography used for purifying tPA include concanavalin A-Sepharose, erythrina trypsin inhibitor (ETI)-Sepharose, anti-tPA IgG antibody and antibody-Sepharose, and fibrin-Sepharose. See, e.g., Rijken and Collen, J. Biol. Chem., 256, 7035-7041 (1981); Heussen et al., J. Biol. Chem. 259, 11635-11638 (1984); Ranby et al., FEBS Lett 146, 289-292 (1982); U.S. Pat. No. 4,505,893. Groups using these chromatography columns have observed a leaching of immobilized proteins from the column.

In addition, these chromatography columns have produced undesired heteroantigenic proteins.

In addition, prior art processes for purifying tPA have required multiple chromatography steps and/or the use of monoclonal antibodies or affinity ligands, such as a plant derived immobilized inhibitor. See, e.g., Rijken et al., Biochimica et Biophysica Acta (BBA)—Protein Structure, 580(1): 140-153, (1979); Einarsson et al., Biochimica et Biophysica Acta (BBA)—Protein Structure and Molecular Enzymology, 830(1): 1-10 (1985); Matsuo et al., Journal of Chromatography A, 369: 391-397 (1986); Vlakh et al., Journal of Biotechnology, 107(3): 275-2845 (2004). In addition, some processes have involved an unfolding step under the influence of altered pHs and temperature, and then involved subsequent refolding. See U.S. Pat. No. 5,158,882.

Thus, a wide variety of purification processes for tPA have been used, but such processes have involved, at least in part, chromatographic procedures involving numerous chromatography steps and/or the use of monoclonal antibodies or affinity ligands, gel filtrations, ammonium sulfate precipitations and the like. All of these procedures greatly decrease the total volume of substantially pure tPA. The inconvenience of running multiple chromatographic columns, for example, increases as the volume of material increases.

In addition, when using prior art methods, truncated tPA may precipitate to a great extent during refolding and subsequent purification steps due to its high hydrophobicity. Furthermore, prior art has indicated that use of arginine in buffers during chromatography is quite difficult because of its viscosity and high conductivity, if the immediate chromatography step is ion exchange. It was understood, for example, that in order to use 1 M arginine in an chromatography buffer, one must apply high pressure to obtain reasonable flow rates. Prior art also has suggested that if one uses ion exchange chromatography to purify a protein recombinantly expressed in E. coli, one should remove arginine from the refolding solution after refolding, for example by diafiltration, to lower the conductivity before loading onto the ion exchange column. Likewise, prior art methods taught away from using 0.3 M or greater concentrations of arginine when purifying recombinant proteins by ion exchange chromatography in light of technical problems associated with a high conductivity of the sample and equilibration buffer, and teachings that ion exchange required lower conductivity to bind the protein.

Previously known methods for purifying tPA, as discussed above, are elaborate, time consuming, and result in a substantial loss of protein due to the involvement of multiple steps in the process. Further, when using previously known methods, tPA precipitates and/or irreversibly bind to resins and membranes during processing because of hydrophobicity in the buffer systems, and therefore high refolding volumes have been required to keep the protein in very dilute conditions.

### SUMMARY OF THE INVENTION

The present invention provides improved processes for purifying recombinant tPA and has overcome problems associated with conventional purification processes of this molecule. In one embodiment, the present invention provides a single step of chromatography for protein purification by altering the arginine concentration in the refolding buffer and in buffers used during chromatography. In this vein, the inventors have discovered optimum arginine concentration ranges for use during chromatography, such as ion exchange chromatography.

Further, the present invention also provides a single step purification for recombinant proteins, such as tPA, found in inclusion bodies upon recombinant expression in bacteria. The present invention reduces the refolding volume needed to isolate biologically active proteins from inclusion bodies, without affecting yield and purity of the protein. The present invention provides a simple, efficient, commercially viable and cost effective process for purifying recombinant tPA for use in patient care.

As discussed above, prior art has suggested that if ion exchange chromatography is used to purify a refolded protein after expression in E. coli, arginine should be removed or reduced from the refolding buffer by diafiltration or dilution to keep conductivity low before loading onto the ion exchange column. Rather than remove arginine from the sample before loading onto the ion exchange column, however, the present invention keeps a relatively high concentration of arginine in buffers and the sample before and during ion exchange chromatography, without loss of the binding efficiency of the ion exchange column. Thus, the present invention avoids loss of protein due to precipitation during purification by keeping a higher arginine concentration and at the same time without hampering the binding to ion exchange resin.

As also discussed above, prior art methods generally purify recombinantly produced tPA (in inclusion bodies in E. coli) using multiple chromatography steps, including lysine affinity chromatography after protein refolding. In the present invention, by contrast, refolded tPA is purified using ion exchange chromatography (such as a SP sepharose column), which is performed in the presence of comparatively high concentration of arginine in samples and buffers. Thus, in one embodiment, the present invention provides a single step cation exchange purification of recombinant proteins, such as tPA produced in E. coli, and eliminates the need for expensive lysine affinity chromatography as well as other additional chromatography steps.

In another embodiment, the present invention provides a single step ion exchange purification of recombinant tPA at a relatively high arginine concentration to maintain solubility of the protein. The present invention provides optimum arginine concentrations in buffers suitable for ion exchange chromatography, for example, at least 0.1 M, or 0.3 M to 0.8 M arginine.

In other embodiments, the present invention reduces the amount of refolding volume required to refold recombinant protein after the protein has been recombinantly expressed in E. coli and formed into inclusion bodies, and after inclusion bodies containing the protein have been solubilized. In one embodiment, the present invention provides a single step purification for recombinant protein, such as tPA, contained in inclusion bodies. The present invention minimizes loss of proteins due to precipitation in a buffer solution during processing by an ion exchanger by keeping arginine concentration relatively high in the buffer solution. In one embodiment, the present invention minimizes the very high dilution of protein needed for refolding efficiency.

In one embodiment, the present invention provides an efficient and improved process for purifying recombinant tPA by performing a single ion exchange chromatography step using arginine at certain concentrations. The present invention also provides optimum arginine concentrations for ion exchange chromatography, such as cation exchange chromatography, when purifying recombinant tPA.

The present method uses certain refolding buffers in processes for purifying bioactive recombinantly expressed protein, such as tPA, from solubilized inclusion bodies after expression in bacteria, such as E. coli. In one embodiment, the method uses a reduced amount of refolding buffer volume, as compared to previously available methods, without affecting yield and purity of the recombinant protein, thereby resulting in higher recovery. For example, in one embodiment, the present method allows the use of less than 35 liters, (e.g., 32 liters) of refolding buffer when processing 5 grams of inclusion bodies as starting material.

In another embodiment, the present invention provides a process that comprises steps for direct refolding of solubilized inclusion bodies without any prior chromatography, thereby minimizing protein loss, reduction of the batch time and cost of chromatography resin and chemicals. In one embodiment, the present invention provides a single step purification for recombinant proteins, such as tPA, that exist in inclusion bodies.

In one embodiment, the present invention provides a process that reduces the amount of refolding buffer volume needed to form active protein after solubilizing inclusion bodies from bacteria used to express the protein, without hampering the refolding efficiency, thereby reducing the capital investment required for a bigger refolding vessel, larger space and the handling and processing problems associated with the use of large volumes.

In one embodiment, the present invention provides a process where protein refolding is performed at room temperature, which reduces refolding cycle time and avoids the costs of cooling. In another embodiment, the refolding and chromatography steps are all performed at room temperature. In another embodiment, the present invention minimizes batch time, both for refolding and recovery of purified protein, thereby increasing yield and purity of tPA.

In one embodiment, the present invention provides a process that gives an industrially feasible process using a reduced amount of refolding buffer when starting from crude starting material, without compromise on the yield and purity.

In another embodiment, the present invention provides a process that uses a single step ion exchange chromatography purification without the need of any affinity chromatography, such as lysine affinity chromatography, directly after refolding.

In one embodiment the present invention provides a process that uses a comparatively higher arginine concentration in the range of 0.1 M to 0.5 molar, such as 0.3 M, which results in higher conductivity for cation exchange chromatography, but lowers the loss of protein due to precipitation.

In one embodiment the present invention provides optimum arginine concentrations in the sample at the start, therefore resulting in initially high starting conductivity without affecting the binding of the material to the ion exchange resin.

In one embodiment, the present invention provides a process for purifying recombinant tPA comprising: (a) recombinantly expressing tPA in cells; (b) isolating inclusion bodies from the cells; (c) solubilizing the inclusion bodies and tPA protein contained therein; (d) refolding the tPA protein in a refolding buffer, wherein the refolding buffer comprises at least 0.1 M arginine, such as at least 0.3 M arginine; (e) thereafter loading the tPA protein onto a chromatography column, such as an ion exchange chromatography column, pre-equilibrated with an equilibrium buffer comprising at least 0.1 M arginine, such as at least 0.3 M arginine; and (f) eluting the tPA protein from the column using (i) the equilibrium buffer and (ii) the equilibrium buffer comprising sodium chloride.

In one embodiment, the process does not comprise performing any chromatography until after refolding in step (d). In another embodiment, the process does not comprise using an affinity chromatography column immediately after the refolding step (d). In another embodiment, the process comprises using only one chromatography column, such as an ion exchange chromatography column, e.g., a cation exchange chromatography column, such as SP Sepharose Fast Flow column.

In certain embodiments, the refolding step (d) is performed at room temperature (23-28° C.). Steps (c)-(f) may also be performed at room temperature (23-28° C.).

In other embodiments, the process further comprises using less than 35 liters of refolding buffer in step (d) for every 5 grams of inclusion bodies solubilized in step (c). In one embodiment, the refolding step occurs in less than 20 hours. In one embodiment, the purity of the eluted tPA protein is at least 96%.

In certain embodiments, the refolding buffer in step (d) comprises 0.3-0.8 M arginine, such as 0.5 M arginine. The refolding buffer may also comprises 0.25 M urea, 0.002-0.004 M reduced glutathione and/or 0.01%-0.05% Tween 80 (w/v). In one embodiment, the refolding buffer in step (d) consists essentially of arginine in the range of 0.3 M to 0.8 M, 150 mM Tris buffer, 2 mM Na-EDTA salt, Tween 80 in the range of 0.01 to 0.05% (w/v), reduced glutathione in the range of 0.2 mM to 4 mM, and urea in the range of 0.25 M to 1 M, wherein the refolding buffer has a pH of about 8.5.

In certain embodiments, the equilibrium buffer comprises 0.1-0.5 M arginine, such as 0.2 M arginine. In other embodiments, the equilibrium buffer comprises sodium citrate and 0.2-0.3 M arginine.

In one embodiment, the purified tPA protein is human truncated tPA.

In one embodiment, the present invention provides a process for purifying recombinant tPA comprising: (a) recombinantly expressing tPA in cells; (b) isolating inclusion bodies from the cells; (c) solubilizing the inclusion bodies and tPA protein contained therein; (d) refolding the tPA protein in a refolding buffer, wherein the refolding buffer comprises at least 0.1 M arginine, such as at least 0.3 M arginine; (e) thereafter loading the tPA protein onto an SP Sepharose column pre-equilibrated with an equilibrium buffer comprising at least 0.1 M arginine, such as at least 0.3 M arginine; and (f) eluting the tPA protein from the column.

In another embodiment, the present invention provides a process for purifying recombinant tPA comprising: (a) recombinantly expressing tPA in cells; (b) isolating inclusion bodies from the cells; (c) solubilizing the inclusion bodies and tPA protein contained therein; (d) refolding the tPA protein in a refolding buffer without performing a chromatography step between step (c) and step (d), wherein the refolding buffer comprises at least 0.3 M arginine; (e) thereafter loading the tPA protein onto a chromatography column pre-equilibrated with an equilibrium buffer comprising 10 to 50 mM sodium citrate and at least 0.2 M arginine, and having a pH between 4 and 5; and (f) eluting the tPA protein from the column using (i) the equilibrium buffer and (ii) the equilibrium buffer comprising sodium chloride.

### BRIEF DESCRIPTION OF THE DRAWINGS

FIG. 1: illustrates the effect of temperature on the refolding cycle time of tPA. Set 8 represents refolding performed at room temperature and set 2 represents refolding performed at 10° C.

FIG. 2: illustrates the effect of arginine concentration on refolding of tPA. The x-axis represents arginine in M.

FIG. 3: illustrates the effect of urea on refolding of tPA.

FIG. 4: illustrates the effect of reduced-state glutathione on refolding of tPA.

FIG. 5: illustrates the effect of Tween 80 concentration on refolding of tPA.

FIG. 6: provides an example purification process flow diagram for purification of recombinant tPA.

### DETAILED DESCRIPTION OF THE PREFERRED EMBODIMENTS

The present invention provides industrially viable processes for purifying tPA, where the processes obviate disadvantages associated with conventional processes of purification. In one embodiment, the present invention provides methods that use a single step of chromatography to purify recombinantly produced protein, such as tPA, by using certain arginine concentrations in relevant buffers. The present invention also presents optimum arginine concentrations for cation exchange chromatography. In one embodiment, the present invention reduces the refolding volume without affecting the yield and purity of the protein.

### DEFINITIONS

The term “tPA” as used herein refers to “tissue plasminogen activator,” which includes a human truncated tissue type plasminogen activator recombinantly produced in E. coli. For example, a truncated human tPA containing amino acids 69-527 of the full length human protein may be produced using recombinant DNA techniques.

The term “room temperature” refers to a temperature range of 23° C. to 28° C.

The term “inclusion bodies” (IB or IBs) refer to nuclear or cytoplasmic aggregates of insoluble substances, such as foreign protein expressed in bacteria (e.g., E. coli). Inclusion bodies may be solubilized, thereby releasing denatured recombinantly produced protein, which then may be refolded to form biologically active protein.

The term “refolding buffer” refers to a buffer used to allow refolding of proteins after they have been solubilized from inclusion bodies previously containing the protein in insoluble form. In one embodiment, the refolding buffer comprises arginine (0.3 to 0.5 M), Tris buffer (150 mM, pH 8.5), Na-EDTA salt (2 mM), Tween 80 (0.01 to 0.05%) (w/v), reduced-state glutathione (0.2 mM to 4 mM), and urea 0.25 M to 1 M, and has a pH of about 8.5.

The term “equilibration buffer” refers to a buffer used to equilibrate a chromatography column, such as an SP Sepharose Fast Flow (SP Sepharose FF) column, used to purify recombinant protein after the protein has been refolded in refolding buffer, after solubilization from inclusion bodies. In one embodiment, the equilibrium buffer is a 10 to 50 mM sodium citrate buffer, pH 4.0 to 5.0 (such as pH 4 or 4.5), containing 0.3 M arginine.

The term “purity” refers to purity as measured by reverse phase HPLC, where samples, for example protein after purification by chromatography, are compared to commercial market standards, such as RETEVASE™. For example, 96% purity means at least 96% purity is achieved by RP-HPLC analysis, as compared to a standard.

The term “arginine” refers to arginine base and/or arginine HCl.

SP Sepharose is cation exchange resin, where the ion exchange group is a sulphopropyl group. The resin is commercially available. For example, SP Sepharose™ Fast Flow (sold by GE Healthcare and Amersham, for example) is a strong cation exchanger with high capacity for proteins of all pI values. The ion exchange group maintains high capacities over a working pH range of 4-13.

In one embodiment of the present invention, processes involve the production and purification of tPA, such as human truncated tPA expressed in E. coli, using known recombinant procedures.

For example, inclusion bodies from E. coli containing the expressed protein may be harvested after bacterial cell lysis. The inclusion bodies are then solubilized using guanidium hydrochloride and DTT. The solubilized mixture is incubated with stirring at a room temperature for 3 to 5 hours. The pH of the mixture is then adjusted to an acidic range and dialyzed against urea. The dialysed sample is then diluted with urea solution and slowly supplemented with Tris and oxidized glutathione. The pH then is adjusted to alkaline around pH 9, and the reaction mixture is again incubated at room temperature with stirring.

In another embodiment, the reaction mixture from above is diluted to 4-8 fold with a refolding buffer, i.e., a buffer that allows recombinant protein solubilized and isolated from the inclusion bodies to refold in an active form. The refolding buffer comprises, for example, arginine, Tris, Na-EDTA salt, Tween 80, urea and reduced glutathione. The protein is incubated in the refolding buffer at a suitable temperature, such as room temperature, for 16-48 hours with stirring. After refolding is complete, the reaction mixture is further diluted with sodium citrate buffer and the pH adjusted to 4 to 5 with urea, and the arginine concentration is maintained at 0.1 to 0.8 M.

The effects of various buffer components and process steps on protein refolding were studied. The present invention presents processes for providing optimum yield when performed at room temperature and the refolding reaction is completed within 16-18 hours.

After inclusion body (IB) solubilization, acidification, dialysis, dilution and refolding, the sample is subjected to a single chromatography step. The chromatography step involves ion exchange, such as cation exchange, using SP based columns, e.g., SP Sepharose chromatography, that is performed using higher arginine concentration. The effective use of relatively higher concentrations of arginine in this process to obtain better yields of purified tPA by reducing protein precipitation is remarkable, because such use is contradictory for ion exchange chromatography due to high conductivity.

In one embodiment, purification of recombinant protein from the refolding reaction mixture is done on a SP Sepharose column, where the column has 80-160 mL resin for proteins isolated from 0.5-1 gm of inclusion bodies, and is pre-equilibrated with sodium citrate buffer and arginine at a pH of 3-5.0.

Samples are loaded onto the column and washed with equilibration buffer. Bound proteins are eluted with a linear gradient of sodium citrate buffer containing 1 M sodium chloride and arginine (e.g., 0.3 M), and sodium citrate buffer having a pH of 4 to 4.5. For example, the protein was eluted with a linear gradient of equilibration buffer (sodium citrate buffer containing 0.3 M arginine), pH 4 to 4.5 and equilibration buffer containing 1 M sodium chloride. The purity of eluted protein was around 96%, as analyzed by RP-HPLC (C-18). tPA activity was determined by Chromozyme tPA (Roche commercial kit).

The process of the present invention has the following advantageous features:
1. Direct refolding after solubilization of inclusion bodies without any prior chromatography thereby minimizing protein loss, reduction of the batch time and cost of chromatography resin and chemicals.
2. Reduction of refolding volume by nearly 35% without hampering refolding efficiency, thereby reducing the capital investment for bigger refolding vessel, larger space requirement and handling and processing problems of large volumes.
3. Refolding at room temperature to reduce the refolding cycle time, and also performing the whole process at room temperature, to avoid the cost of cooling.
4. Industrially feasible processes using a reduced amount of refolding buffer with regard to crude starting material, without compromising the yield and purity.
5. Using a single step ion exchange chromatography purification without the need of any affinity chromatography directly after refolding.
6. Using comparatively higher arginine concentrations in the range of 0.1-0.8 M, such as 0.5 M, though results in higher conductivity for cation exchange chromatography, does not hamper the binding of protein and lowers the loss of protein due to precipitation.
7. To provide optimum arginine concentrations in samples, resulting in an initially high start conductivity in those samples, but without affecting the binding of the material to the ion exchange resin.

The following examples are provided to demonstrate certain embodiments of the invention. Those of skill in the art will appreciate that the techniques disclosed in the examples represent methods discovered by the inventors to function well in the practice of the invention, and thus are considered illustrative modes for its practice. Those of skill in the art will, in light of the present disclosure, appreciate that changes can be made in the specific embodiments disclosed below, and one can obtain like or similar results without departing from the spirit and scope of the invention.

### Example 1

### Preparation of tPA

Human truncated tPA (i.e., amino acids 69-527 of the full length human protein) was recombinantly expressed in E. coli using available techniques and DNA constructs. Inclusion bodies containing recombinantly expressed tPA protein were harvested after bacterial cell lysis, and solubilized in 6 M to 8 M (i.e., 8 M in this Example) guanidium hydrochloride (1:30 w/v) in 200-300 mM (i.e., 200 mM here) DTT by incubating with stirring at room temperature for 3 hours. Thereafter, the mixture was adjusted to pH 3 with hydrochloric acid (conc) and dialyzed overnight in 6-8 M (i.e., 8 M here) urea at 10-15° C. The dialyzed sample was then diluted 10 to 20 fold with 6-8 M urea (i.e., 8 M here) and gradually supplemented with Tris (to a final concentration 50 mM) and oxidized glutathione (to a final concentration 25 mM) after adjusting the pH to 9.3 with 3 M sodium hydroxide solution, and then incubated for 3-5 hours at room temperature with stirring.

### Example 2

### Refolding of tPA Protein after Solubilization

The sample was then diluted 4 to 8 fold with a refolding buffer having a pH of 8.5, and containing arginine (0.5 M) Tris buffer (150 mM, pH 8.5), Na-EDTA salt (2 mM), Tween 80 (0.05%) (w/v), reduced-state glutathione (0.2 mM) and urea (0.25M), and incubated at temperatures between 10° C. to room temperature (RT) for 16-48 hours with stirring. After incubation in the refolding buffer, the sample was diluted with 10 to 50 mM (i.e., 20 mM here) sodium citrate buffer, pH 4.0 to 5.0 (pH 4.0 here), containing 1 M urea to bring the final arginine concentration down to 0.3 M and to adjust the pH to 4.0 to 5.0 (pH 4.5 here).

### Example 3

### Purification

The sample was then loaded on a SP Sepharose FF (Amersham) column (80 to 160 ml resin used for proteins from 0.5 to 1 gm of IBs) pre-equilibrated with equilibrium buffer of 10 to 20 mM sodium citrate buffer containing 0.3 M arginine, having a pH 4.5. After sample loading, the column was washed with 5 to 10 column volumes (CV) of equilibration buffer and bound proteins were eluted with a linear gradient (30 CV) or step gradients of equilibration buffer (Elution Buffer A) and equilibration buffer containing 1 M sodium chloride (Elution Buffer B). The purity achieved as evidenced by RP-HPLC (C-18) was not less than 98%. The protein was fully active as determined by Chromzyme tPA activity assay (Roche).

### RP-HPLC: C-18 Reverse Phase Analytical HPLC Column

In a reverse phase chromatography, the stationary phase is polar and the mobile phase used for elution has increasing non-polarity, which helps elute proteins based on their hydrophobicity. A C-18 reverse phase column (5 μm bed size, 300 A pore size, 25 cm in length) is connected to an automated HPLC system and equilibrated with solvent A (0.1% TFA in 10% acetonitrile in H₂O) at 0.8 ml/min. After the baseline at 220 nm is stabilized, the protein sample is injected onto the column (1-15 μg) and proteins are eluted from the column with a gradient of acetonitrile containing solvent B (0.1% TFA in 100% acetonitrile in H₂O). Protein that elutes from the column is detected at 220 nm.

### Example 4

### Example Temperature, Volumes, Buffers and Time Periods Regarding Steps in tPA Purification Process, as Described in Examples 1-3

tPA: DOWN STREAM PROCESS
FLOW DIAGRAM (5 grams IB)

| | Start Time | End Time | Vol | Temp |
|---|---|---|---|---|
| | (Hrs) | (Hrs) | (L) | (° C.) |
| IB Solubilization | 00 | 03 | 0.30 | RT |
| ↓ | | | | |
| Acidification | 03 | 15 | 0.32 | RT |
| (pH 3.0) and | | | | |
| Dialysis (8M Urea) | | | | |
| ↓ | | | | |
| Mixed disulfide | 15 | 18 | 8.0 | RT |
| reaction | | | | |
| ↓ | | | | |
| Refolding | 18 | 34 | 64.0 | RT |
| ↓ | | | | |
| Dilution | 34 | 37 | 12.0 | RT |
| ↓ | | | | |
| SP-Sepharose | 41 | 46 | 0.5 | RT |
| (100 ml resin) | | | | |

(IB: Inclusion bodies; RT: Room temperature (25-28° C.);

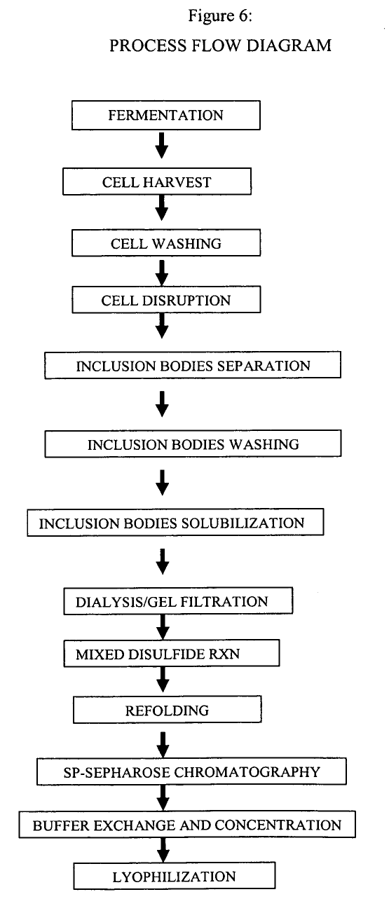

See also FIG. 6.

### Example 5

### Studies of Various Components on Refolding Efficiency

### A) Effect of Temperature on Refolding

Following the methods as described in Example 2, refolding experiments were done at various temperatures, and the effects of conventional incubation temperature (i.e., 10° C.) and room temperature on activity and yield of purified tPA protein was examined. Specifically, the enzymatic activity of tPA was determined by means of a Chromozyme tPA assay (Roche Diagnostics) and quantified with respect to a standard curve of native tPA to provide a measure for renaturation/activity. The yield in mIU/ml of purified tPA protein, after 16 hours of refolding at room temperature (23-25° C.) was compared to that at done at 10° C.

TABLE 1

Set

| | 1 | 2 |
|---|---|---|
| | Conc | Conc |
| Arginine (M) | 0.5 | 0.5 |
| Tris buffer (M) | 0.15 | 0.15 |
| Na-EDTA salt (0.2M) | 0.002 | 0.002 |
| Tween 80 (%, W/V) | 0.05 | 0.05 |
| Glutathione reduced (M) | 0.002 | 0.002 |
| Urea (M) | 0.25 | 0.25 |
| pH | 8.5 | 8.5 |
| Buffer Vol make up (ml) | 17.5 | 17.5 |
| Sample Vol (ml) | 2.5 | 2.5 |
| Incubation (° C.) | 10 | RT |

| Activity (mIU/ml) | Absorbance | mIU/ml | Abs | mIU/ml |
|---|---|---|---|---|
| 0 Hrs. | 0.254 | 0.7 | 0.249 | 0.7 |
| 18 Hrs. | 0.848 | 2.5 | 1.241 | 3.686 |
| 24 Hrs. | 0.936 | 2.73 | 1.311 | 3.896 |
| 42 Hrs. | 0.912 | 2.64 | 1.137 | 3.375 |

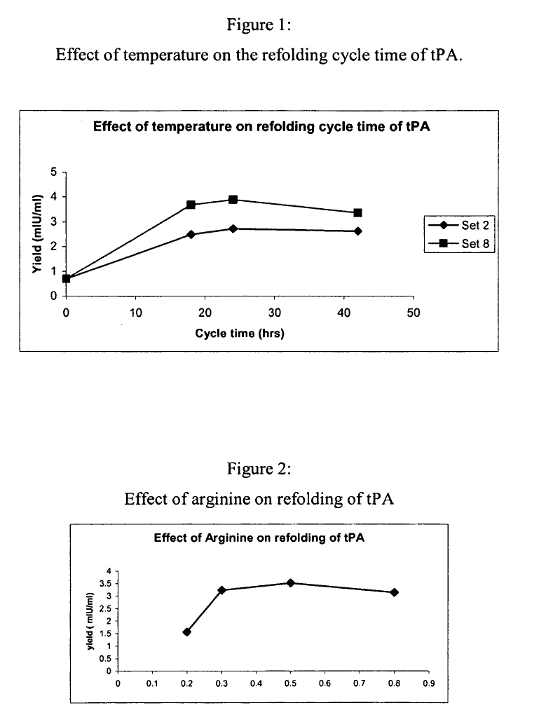

As seen in the table above and in FIG. 1, the refolding reaction progressed faster and resulted in a higher yield when performed at room temperature (23-25° C.), as compared to 10° C.

### B) Effect of Arginine Concentration in Refolding Buffer on tPA Yield

Following the methods described in Example 2, the effect of different arginine concentrations in the refolding buffer on tPA refolding (i.e., active protein) yield was studied, after supplementing the refolding buffer (as described in Example 2) with different concentrations of arginine. Refolding efficiency (i.e., biological activity) was measured by a Chromozyme tPA activity assay kit (Roche), which measured active tPA in milli IU (international units) per mL. The effect of 0.2-1.0 M arginine in the refolding buffer on tPA refolding (i.e., biological activity of tPA) was studied, the results of which are presented below.

| Arg (M) | yield (mIU/mL) |
|---|---|
| 0.2 | 1.56 |
| 0.3 | 3.228 |
| 0.5 | 3.519 |
| 0.8 | 3.1382 |

The above results, as well as FIG. 2, show that refolding efficiency was highest in refolding buffer containing 0.5 M arginine, as this concentration produced the highest amount of active tPA in milli IU per mL. The most effective concentrations of arginine for refolding of tPA occurred in the range of about 0.3 M to 0.8 M arginine.

### C) Effect of Urea Concentration in Refolding Buffer on tPA Yield

Following the methods described in Example 2, the effect of the urea concentration in the refolding buffer on tPA refolding (i.e., active protein) yield was studied after supplementing the refolding buffer (as described in Example 2) with different concentrations of urea. Refolding efficiency was measured by a Chromozyme tPA activity assay kit (Roche), which measured active tPA in milli IU (international units) per mL. The effect of 0 to 1 M urea in the refolding buffer on tPA refolding (i.e., biological activity of tPA) was studied, the results of which are presented below.

| | Yield |
|---|---|
| Urea (M) | (mIU/ml) |
| 0 | 2.6 |
| 0.25 | 3.686 |
| 0.5 | 3.31 |
| 0.75 | 3.29 |
| 1 | 3.23 |

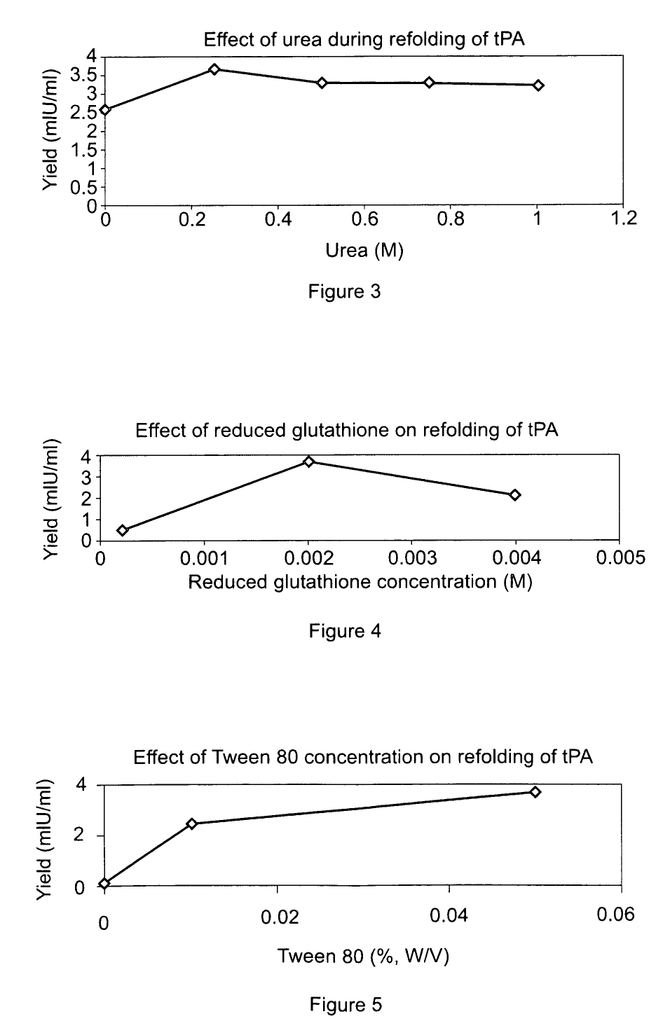

The above results, as well as FIG. 3, show that refolding efficiency was highest in refolding buffer containing 0.25 M urea, as this concentration produced the highest amount of active tPA in milli IU per mL.

### D) Effect of Reduced Glutathione Concentration in Refolding Buffer on tPA Yield

Following the methods described in Example 2, the effect of reduced-state glutathione concentration in the refolding buffer on tPA refolding (i.e., active protein) yield was studied after supplementing the refolding buffer (as described in Example 2) with different concentrations of reduced glutathione. Refolding efficiency was measured by a Chromozyme tPA activity assay kit (Roche), which measured active tPA in milli IU (international units) per mL. The effect of 0.0002 to 0.004 M reduced glutathione in the refolding buffer on tPA refolding (i.e., biological activity of tPA) was studied, the results of which are presented below.

| Reduced | Yield |
|---|---|
| Glutathione (M) | (mIU/ml) |
| 0.0002 | 0.5 |
| 0.002 | 3.686 |
| 0.004 | 2.127 |

The above results, as well as FIG. 4, show that refolding efficiency was highest in refolding buffer containing 0.002 M reduced glutathione, as this concentration produced the highest amount of active tPA in milli IU per mL.

### E) Effect of Tween 80 Concentration in Refolding Buffer on tPA Yield

Following the methods described in Example 2, the effect of Tween 80 concentration in the refolding buffer on tPA refolding (i.e., active protein) yield was studied after supplementing the refolding buffer (as described in Example 2) with different concentrations of Tween 80. Refolding efficiency was measured by a Chromozyme tPA activity assay kit (Roche), which measured active tPA in milli IU (international units) per mL. The effect of 0% to 0.05% Tween 80 (w/v) in the refolding buffer on tPA refolding (i.e., biological activity of tPA) was studied, the results of which are presented below.

| | Yield |
|---|---|
| Tween 80% (w/v) | (mIU/ml) |
| 0 | 0.1 |
| 0.01 | 2.51 |
| 0.05 | 3.686 |

The above results, as well as FIG. 5, show that refolding efficiency was highest in refolding buffer containing 0.05% Tween 80 (w/v), as this concentration produced the highest amount of active tPA in milli IU per mL.

All of the compositions and methods disclosed and claimed herein can be made and executed without undue experimentation in light of the present disclosure. While the compositions and methods of this invention have been described in terms of certain embodiments, those of skill in the art will understand that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results will be achieved. All such similar substitutes and modifications apparent to those skilled in the art are deemed to be within the spirit, scope and concept of the invention as defined by the invention.

What is claimed is:

1. A process for purifying recombinant tissue plasminogen activator (tPA) comprising:
(a) recombinantly expressing tPA in cells;
(b) isolating inclusion bodies from the cells;
(c) solubilizing the inclusion bodies and tPA protein contained therein;
(d) refolding the tPA protein in a refolding buffer, wherein the refolding buffer comprises at least 0.1 M arginine;
(e) thereafter loading the tPA protein onto an ion exchange chromatography column pre-equilibrated with an equilibrium buffer comprising at least 0.1 M arginine; and
(f) eluting the tPA protein from the column using (i) the equilibrium buffer and (ii) the equilibrium buffer comprising sodium chloride.

2. The process of claim 1, wherein the process does not comprise performing any chromatography until after step (d).

3. The process of claim 1, wherein the process does not comprise using an affinity chromatography column immediately after the refolding step (d).

4. The process of claim 1, wherein the refolding step (d) is performed at room temperature (23-28° C.).

5. The process of claim 1, wherein steps (c)-(f) are performed at room temperature (23-28° C.).

6. The process of claim 1, wherein the process further comprises using less than 35 liters of refolding buffer in step (d) for every 5 grams of inclusion bodies solubilized in step (c).

7. The process of claim 1, wherein the refolding step occurs in less than 20 hours.

8. The process of claim 1, wherein purity of the eluted tPA protein is at least 96%.

9. The process of claim 1, wherein the refolding buffer in step (d) comprises 0.3-0.8 M arginine.

10. The process of claim 1, wherein the refolding buffer in step (d) comprises 0.5 M arginine.

11. The process of claim 1, wherein the refolding buffer in step (d) comprises 0.25 M urea.

12. The process of claim 1, wherein the refolding buffer in step (d) comprises 0.002-0.004 M reduced glutathione.

13. The process of claim 1, wherein the refolding buffer in step (d) comprises 0.002 M reduced glutathione.

14. The process of claim 1, wherein the refolding buffer in step (d) comprises 0.01%-0.05% Tween 80 (w/v).

15. The process of claim 1, wherein the refolding buffer in step (d) consists essentially of arginine in the range of 0.3 M to 0.8 M, 150 mM Tris buffer, 2 mM Na-EDTA salt, Tween 80 in the range of 0.01 to 0.05% (w/v), reduced glutathione in the range of 0.2 mM to 4 mM, and urea in the range of 0.25 M to 1 M, wherein the refolding buffer has a pH of about 8.5.

16. The process of claim 1, wherein the equilibrium buffers in steps (e) and (f) comprise 0.1-0.5 M arginine.

17. The process according to claim 1, wherein the equilibrium buffers in steps (e) and (f) comprise sodium citrate and 0.2-0.3 M arginine.

18. The process according to claim 1, wherein the equilibrium buffers in steps (e) and (f) comprise 0.2 M arginine.

19. The process of claim 1, wherein the purified tPA protein is human truncated tPA.

20. The process of claim 1, wherein the process further comprises using only one chromatography column.

21. The process of claim 1, wherein the ion exchange chromatography column in steps (e) and (f) is a cation exchange chromatography column.

22. The process of claim 1, wherein the ion exchange chromatography column in steps (e) and (f) is a SP Sepharose Fast Flow column.

23. A process for purifying recombinant tissue plasminogen activator (tPA) comprising:
(a) recombinantly expressing tPA in cells;
(b) isolating inclusion bodies from the cells;
(c) solubilizing the inclusion bodies and tPA protein contained therein;
(d) refolding the tPA protein in a refolding buffer, wherein the refolding buffer comprises at least 0.1 M arginine;
(e) thereafter loading the tPA protein onto a SP Sepharose column pre-equilibrated with an equilibrium buffer comprising at least 0.1 M arginine; and
(f) eluting the tPA protein from the column.

24. A process for purifying recombinant tissue plasminogen activator (tPA) comprising:
(a) recombinantly expressing tPA in cells;
(b) isolating inclusion bodies from the cells;
(c) solubilizing the inclusion bodies and tPA protein contained therein;
(d) refolding the tPA protein in a refolding buffer without performing a chromatography step between step (c) and step (d), wherein the refolding buffer comprises at least 0.3 M arginine;
(e) thereafter loading the tPA protein onto a chromatography column pre-equilibrated with an equilibrium buffer comprising 10 to 50 mM sodium citrate and at least 0.2 M arginine, and having a pH between 4 and 5; and
(f) eluting the tPA protein from the column using (i) the equilibrium buffer and (ii) the equilibrium buffer comprising sodium chloride.
